(19) United States
(12) Patent Application Publication
(10) Pub. No.: US 2026/0256856 A1
(43) Pub. Date: Sep. 3, 2026

(54) COMPOSITION

(71) Applicant: HONDA MOTOR CO., LTD., Tokyo (JP)

(72) Inventors: Nozomi SHIOBARA, Saitama (JP); Shohei KINOSHITA, Saitama (JP); Mizuho DOI, Saitama (JP); Kenji MACHIDA, Saitama (JP); Minoru GOTO, Saitama (JP)

(21) Appl. No.: 19/159,617

(22) PCT Filed: Feb. 2, 2024
(86) PCT No.: PCT/JP2024/003440

§ 371 Date: Aug. 26, 2025

(30) Foreign Application Priority Data
Mar. 2, 2023 (JP) 2023-031565

(51) Int. Cl. A61K 36/05; A61P 1/04

(52) U.S. Cl. CPC A61K 36/05; A61P 1/04

(57) ABSTRACT

Provided is a composition containing Chlamydomonas reinhardtii or an extract of Chlamydomonas reinhardtii, wherein the Chlamydomonas reinhardtii or the extract of the Chlamydomonas reinhardtii contains an antagonist of an histamine receptor.

### TECHNICAL FIELD

The present invention relates to a composition including Chlamydomonas reinhardtii or an extract of Chlamydomonas reinhardtii.

### BACKGROUND ART

Pharmaceutical products are being developed to ensure healthy lives and promote well-being for all people of all ages.

Patent Document 1 discloses a pharmaceutical composition including a wound healing substance. Here, the wound healing substance is selected from the group of vitamins or provitamins, carotinoids, analgesics, disinfectants, haemostatic agents, antihistamines, antimicrobial metals or salts thereof, plant-derived wound healing substances or mixtures of the substances, plant extracts, enzymes, growth factors, enzyme inhibitors, and a combination thereof.

On the other hand, Patent Document 2 discloses Honda DREAMO strain (Accession No. FERM BP-22306) as an example of the Chlamydomonas reinhardtii.

### CITATION LIST

### Patent Document

Patent Document 1: Japanese Unexamined Patent Application (Translation of PCT Application), Publication No. 2009-519917
Patent Document 2: PCT International Publication No. WO2017/217116

### DISCLOSURE OF THE INVENTION

### Problems to be Solved by the Invention

However, it is not known that the Chlamydomonas reinhardtii or extracts of Chlamydomonas reinhardtii function as antagonists of histamine receptors.

An object of the present invention is to provide a composition containing, as an active ingredient, Chlamydomonas reinhardtii or an extract of Chlamydomonas reinhardtii that functions as an antagonist of histamine receptors.

### Means for Solving the Problems

(1) A composition including Chlamydomonas reinhardtii or an extract of Chlamydomonas reinhardtii, in which the Chlamydomonas reinhardtii or the extract of Chlamydomonas reinhardtii includes an antagonist of a histamine receptor.

(2) The composition as described in (1), in which the Chlamydomonas reinhardtii is a Honda DREAMO strain (Accession No. FERM BP-22306).

(3) The composition as described in (1) or (2), which is a therapeutic agent for peptic ulcers, a therapeutic agent for reflux esophagitis, a therapeutic agent for upper gastrointestinal bleeding, or a therapeutic agent for gastric mucosal lesions.

(4) The composition as described in any one of (1) to (3), in which the histamine receptor is histamine receptor H₂.

(5) The composition as described in any one of (1) to (4), in which the Chlamydomonas reinhardtii is a freshwater alga.

### Effects of the Invention

According to the present invention, it is possible to provide a composition containing, as an active ingredient, Chlamydomonas reinhardtii or an extract of Chlamydomonas reinhardtii that functions as an antagonist of histamine receptors.

### PREFERRED MODE FOR CARRYING OUT THE INVENTION

Hereinafter, embodiments of the present invention will be described.

The composition of this embodiment includes Chlamydomonas reinhardtii or an extract of Chlamydomonas reinhardtii, and the Chlamydomonas reinhardtii or the extract of Chlamydomonas reinhardtii includes an antagonist of a histamine receptor.

Examples of the Chlamydomonas reinhardtii include, but are not particularly limited to, UTEX 90 strain and Honda DREAMO strain (Accession No. FERM BP-22306). Here, the UTEX 90 strain is the original strain of the Honda DREAMO strain.

Preferably, the Chlamydomonas reinhardtii is a freshwater alga. As a result, a large amount of culture can be performed on land.

The histamine receptors include histamine receptor H₁, histamine receptor H₂, histamine receptor H₃, and histamine receptor H₄. Among these, histamine receptor H₂ is preferable.

The composition of the present embodiment can be applied to pharmaceutical products such as a therapeutic agent for peptic ulcers, a therapeutic agent for reflux esophagitis, a therapeutic agent for upper gastrointestinal bleeding, a therapeutic agent for gastric mucosal lesions or the like, because it contains, as an active ingredient, Chlamydomonas reinhardtii or an extract of Chlamydomonas reinhardtii that functions as an antagonist of a histamine receptor. Peptic ulcers include, for example, gastric ulcers, duodenal ulcers, anastomotic ulcers, a Zollinger-Ellison syndrome. Examples of the gastric mucosal lesions include an acute gastric mucosal lesion and a chronic gastric mucosal lesion.

Administration forms of the pharmaceutical products may be either oral administration (internal use) or parenteral administration (external use, injection).

Dosage forms of the pharmaceutical products are not particularly limited, and examples thereof include solid preparations such as tablets, granules, powders, capsules, etc.; liquid preparations such as solutions, suspensions, emulsions, etc.; lyophilized preparations and the like.

The pharmaceutical products can be manufactured using known methods.

The composition of the present embodiment may be applied to, for example, quasi-pharmaceutical products, cosmetics, or foods.

The composition of the present embodiment is preferably used for humans, but may be used for animals other than humans.

A culture method of the Chlamydomonas reinhardtii is not particularly limited, and examples thereof include a static culture method, a shaking culture method, a deep culture method, and an aeration culture method.

The Chlamydomonas reinhardtii can be cultured in large quantities on land by culturing outdoors. A method of culturing the Chlamydomonas reinhardtii outdoors is not particularly limited, and examples thereof include a method of culturing the Chlamydomonas reinhardtii in an open pond system using a raceway bioreactor, a method of culturing the Chlamydomonas reinhardtii using a flat panel photobioreactor installed outdoors, and the like.

A medium used for culturing the Chlamydomonas reinhardtii is not particularly limited, and examples thereof include a TAP medium, a urea medium, an HSM medium, and a BG11 medium.

A temperature during culturing the Chlamydomonas reinhardtii is not particularly limited, but is, for example, 2° C. or higher and 38° C. or lower.

A light and dark cycle when culturing the Chlamydomonas reinhardtii is not particularly limited, and is, for example, a 24-hour cycle, in which a light phase is 6 hours or more and 24 hours or less.

A photosynthesis effective photon flux density in the light phase is not particularly limited, but is, for example, 50 μmol/m²/s or more and 2,000 μmol/m²/s or less.

A gas aeration amount per unit volume when culturing the Chlamydomonas reinhardtii by the aeration culture method is not particularly limited, but is, for example, 0.001 VVM or more and 10 VVM or less. It should be noted that VVM indicates how many times the volume ratio of the gas with respect to the medium is aerated in one minute.

Carbon dioxide is preferably supplied to the medium when culturing the Chlamydomonas reinhardtii by the aeration culture method. A concentration of carbon dioxide in the gas to be aerated is not particularly limited, but is, for example, 0.1% by volume or more and 10% by volume or less. A supply source of carbon dioxide is not particularly limited, and examples thereof include a gas discharged from a carbon dioxide cylinder, a factory, or the like, and concentrated carbon dioxide recovered from the atmosphere by direct air capture (DAC).

A method for recovering the cultured Chlamydomonas reinhardtii is not particularly limited, and examples thereof include a centrifugation method, a filtration method, or the like.

The recovered Chlamydomonas reinhardtii may be dried as necessary.

A method for drying the Chlamydomonas reinhardtii is not particularly limited, and examples thereof include a freeze drying method, a spray drying method, a hot air drying method, a sun drying method, and the like.

An extraction method used for extracting the Chlamydomonas reinhardtii is not particularly limited, and examples thereof include an ultrasonic extraction method.

A solvent used for extraction of the Chlamydomonas reinhardtii is not particularly limited, and examples thereof include lower alcohols such as methanol, ethanol, propyl alcohol, isopropyl alcohol, etc.; lower aliphatic ketones such as acetone, methyl ethyl ketone, etc.; polyhydric alcohols such as 1,3-butylene glycol, propylene glycol, glycerin, etc.; and hydrophilic organic solvents such as dimethyl sulfoxide (DMSO), etc. Among these, DMSO is preferable.

A method for recovering an extract solution of Chlamydomonas reinhardtii is not particularly limited, and examples thereof include a centrifugal separation method, a filtration method, etc.

The recovered extract of Chlamydomonas reinhardtii may be dried as necessary.

A method for drying the extract of Chlamydomonas reinhardtii is not particularly limited, and examples thereof include a freeze-drying method, etc.

### EXAMPLES

Hereinafter, examples of the present invention will be described, but the present invention is not limited to the examples.

### Example 1

[Culture of Chlamydomonas reinhardtii]

Using 2 L of a TAP medium, Honda DREAMO strain as the Chlamydomonas reinhardtii was statically cultured (seed culture) for 5 days under the culture conditions shown below.
Temperature: 25° C.
Light and Dark Cycle: 12 hours of light phase and 12 hours of dark phase
Photosynthesis effective photon flux density in the light phase: 60 to 80 μmol/m²/s
Light Source: white LED

Next, 500 mL of the seed culture product was centrifuged at 3000 rpm for 5 minutes, the culture supernatant was removed, and the seed-cultured Honda DREAMO strain was recovered.

Next, the recovered Honda DREAMO strain was statically cultured (pre-culture) for 7 days using 2 L of the TAP medium under the same culture conditions as in the seed culture.

Next, aeration culture of the pre-cultured Honda DREAMO strain was carried out for 7 days by using 5 L of a urea medium (main culture) under the same culture conditions as in the seed culture. The aeration conditions were as follows.
Gas aeration per unit volume: 0.15 VVM
Concentration of carbon dioxide in the gas: 5 vol %

Next, 5 L of the main culture product was centrifuged at 6,000 rpm for 10 minutes, the culture supernatant was removed, and the main cultured Honda DREAMO strain was recovered.

### [Extraction]

Approximately 20 mL of the recovered Honda DREAMO strain was sonicated with 100 mL of DMSO for 5 minutes using an ultrasonic homogenizer. At this time, ultrasonic extraction was performed on ice.

Next, a mixture of an extract solution and a residue was centrifuged at 3,000 rpm for 5 minutes, the residue was removed, and about 100 mL of the extract solution was recovered.

Next, the extract solution was freeze-dried to obtain 490.74 mg of the extract of Honda DREAMO strain.

### [Preparation of Analyte]

Using an ultrasonic homogenizer, 490 mg of the Honda DREAMO strain extract was dissolved in 4.9 mL of DMSO to obtain a DMSO solution of 100 mg/mL of the Honda DREAMO extract.

Next, the DMSO solution of the Honda DREAMO strain extract was centrifuged at 15,000 rpm for 5 seconds, and the supernatant was collected to obtain an analyte.

Next, in order to determine the concentration of the analyte to be used in the assay (in vitro functionality evaluation test), the following two tests were carried out.

### [Cell Proliferation Inhibition Test]

The analyte was added to HEK293 cells (human embryonic kidney cell-derived cell line) grown in D-MEM (High Glucose)+10% FBS+1% Penicillin—Streptomycin medium at 13 different concentrations (0, 10, 20, 30, 40, 50, 60, 70, 80, 90, 100, 250, and 500 μg/mL), and a dose-dependent test was carried out. The medium was replaced with a fresh one 24 hours after the sample was added to the cells and the cells were stained with WST-8 for 4 hours. Next, the optical density (O.D.) at a wavelength of 450 nm was measured using a FlexStation 3 multimode microplate reader (manufactured by Molecular Devices). Here, the test was performed with n=3.

As a result, no decrease in the optical density was observed at any concentration of the analyte, and therefore, it was determined that there was no influence due to the concentration.

### [Effect of Analyte on Optical Density]

The analyte was added to the D-MEM (High Glucose)+10% FBS+1% Penicillin—Streptomycin medium at 13 different concentrations (0, 10, 20, 30, 40, 50, 60, 70, 80, 90, 100, 250, and 500 μg/mL), the medium was replaced with a fresh one 24 hours after the addition, and the sample was stained with WST-8 for 4 hours. Next, the optical density at a wavelength of 450 nm was measured using a FlexStation 3 multimode microplate reader (manufactured by Molecular Devices). Here, the test was performed with n=3.

As a result, an increase in the optical density was observed when the concentration of the analyte was 250 μg/mL and 500 μg/mL. Therefore, it was found that the addition of the analyte at 250 μg/mL and 500 μg/mL affected the optical density irrespective of the cells. On the other hand, it was seen that the optical density was not affected when the analyte was added at a concentration of 100 μg/mL or less.

From the above results, 100 μg/mL, which is the maximum value of the concentration at which the cell proliferation inhibition and the influence on the optical density of the analyte were not observed, was defined as the analyte concentration in the assay.

### [Assays]

An assay of an analyte that targets histamine receptor H₂ as a molecular target was performed at an analyte concentration of 100 μg/mL. Here, the number of assay repeats was two. Note that the assay was outsourced to Eurofins Pharma Discovery Services and was performed in two assay systems to exclude false positives. Here, a molecular target having an inhibition ratio of 50% or more was judged as having efficacy as an antagonist.

Table 1 shows the results of the analyte assay.

TABLE 1

| | | | Efficacy of pharmaceutical |
|---|---|---|---|
| Targeted | | Inhibition | product targeting the |
| molecule | Action | ratio [%] | targeted molecule |
| Histamine | Antag- | 54 | Therapeutic agent for peptic ulcers, |
| receptor H₂ | onist | | therapeutic agent for reflux |
| | | | esophagitis, therapeutic agent for |
| | | | upper gastrointestinal bleeding, |
| | | | therapeutic agent for gastric |
| | | | mucosal lesions. |

From Table 1, it can be seen that the Honda DREAMO strain extract contained in the analyte contained an antagonist of the histamine receptor H₂, that is, the Honda DREAMO strain extract functioned as an antagonist of the histamine receptor H₂.

1. A composition comprising Chlamydomonas reinhardtii or an extract of Chlamydomonas reinhardtii,
wherein the Chlamydomonas reinhardtii or the extract of Chlamydomonas reinhardtii comprises an antagonist of a histamine receptor.

2. The composition according to claim 1, wherein the Chlamydomonas reinhardtii is a Honda DREAMO strain (Accession No, FERM BP-22306).

3. The composition according to claim 1, which is a therapeutic agent for peptic ulcers, a therapeutic agent for reflux esophagitis, a therapeutic agent for upper gastrointestinal bleeding, or a therapeutic agent for gastric mucosal lesions.

4. The composition according to claim 1, wherein the histamine receptor is histamine receptor H₂.

5. The composition according to claim 1, wherein the Chlamydomonas reinhardtii is a freshwater alga.
